(19) United States
(12) Patent Application Publication
(10) Pub. No.: US 2022/0299483 A1
(43) Pub. Date: Sep. 22, 2022

(54) STATE ESTIMATION DEVICE, STATE ESTIMATION METHOD, AND RECORDING MEDIUM

(71) Applicant: NEC Corporation, Minato-ku, Tokyo (JP)

(72) Inventor: Daisuke SUGII, Tokyo (JP)

(73) Assignee: NEC Corporation, Minato-ku, Tokyo (JP)

(21) Appl. No.: 17/642,347

(22) PCT Filed: Sep. 10, 2020
(86) PCT No.: PCT/JP2020/034249

§ 371 Date: Mar. 11, 2022

(30) Foreign Application Priority Data
Sep. 25, 2019 (JP) 2019-173795

(51) Int. Cl. G01N 29/44; G01N 29/04; G01N 33/02

(52) U.S. Cl. CPC G01N 29/4481; G01N 29/4418; G01N 29/04; G01N 33/025

(57) ABSTRACT

The state estimation device includes a generation unit and an estimation unit. The generation unit applies an acoustic stimulus to an object and generates an impulse response of the object. The estimation unit estimates the state of the object by deep learning using the learned model, based on the impulse response.

### TECHNICAL FIELD

The present disclosure relates to a technique for estimating the state of an object using acoustic stimulation.

### BACKGROUND ART

As a technique to judge the afterripening degree and the good state for eating of fruit/vegetable, there is known a technique of checking the reaction by hitting an object like a watermelon. Further, rather than hitting by a person, Patent Document 1 proposes a method of determining the grade or the like of the fruit/vegetable by giving impacts on the fruit/vegetable by the impact unit and detecting the vibration wave emitted by the fruit/vegetable.

### PRECEDING TECHNICAL REFERENCES

### Patent Document

Patent Document 1: Japanese Patent application Laid-Open under No. 1-231975

### SUMMARY

### Problem to be Solved by the Invention

In the above technique, there is a problem that decay and deterioration tend to progress from the point where the impact is given to the fruit/vegetable.

It is an object of the present disclosure to provide a state estimation method capable of estimating the state of an object without causing deterioration or damage to the object.

### Means for Solving the Problem

According to one aspect of the present disclosure, there is provided a state estimation device comprising:

a generation unit configured to apply an acoustic stimulus to an object to generate an impulse response of the object; and

an estimation unit configured to estimate a state of the object based on the impulse response, by deep learning using a learned model.

According to another aspect of the present disclosure, there is provided a state estimation method comprising:

applying an acoustic stimulus to an object to generate an impulse response of the object; and

estimating a state of the object based on the impulse response, by deep learning using a learned model.

According to still another aspect of the present disclosure, there is provided a recording medium recording a program which causes a computer to execute a process of:

applying an acoustic stimulus to an object to generate an impulse response of the object; and

estimating a state of the object based on the impulse response, by deep learning using a learned model.

### Effect of the Invention

According to the present invention, it is possible to estimate the state of an object without causing deterioration or damage to the object.

### BRIEF DESCRIPTION OF THE DRAWINGS

FIG. 1 shows an overall configuration of an inspection apparatus according to the first example embodiment.

FIG. 2 shows a functional configuration of a CPU.

FIG. 3 shows an image of a short-time power signal generated by an impulse response generating unit.

FIGS. 4A and 4B are display examples of the estimation result of the afterripening degree of fruit/vegetable.

FIGS. 5A and 5B are display examples of the estimation result of the afterripening degree of fruit/vegetable.

FIG. 6 is a flowchart of a learning process by an inspection apparatus.

FIG. 7 is a flowchart of a determination process by the inspection apparatus.

FIG. 8 shows a functional configuration of the state estimation device according to the second example embodiment.

### EXAMPLE EMBODIMENTS

Hereinafter, example embodiments of the present disclosure will be described with reference to the accompanying drawings.

### First Example Embodiment

First, a first example embodiment of the present disclosure will be described. The first example embodiment applies the present disclosure to an inspection apparatus for estimating the afterripening degree of fruit/vegetable.

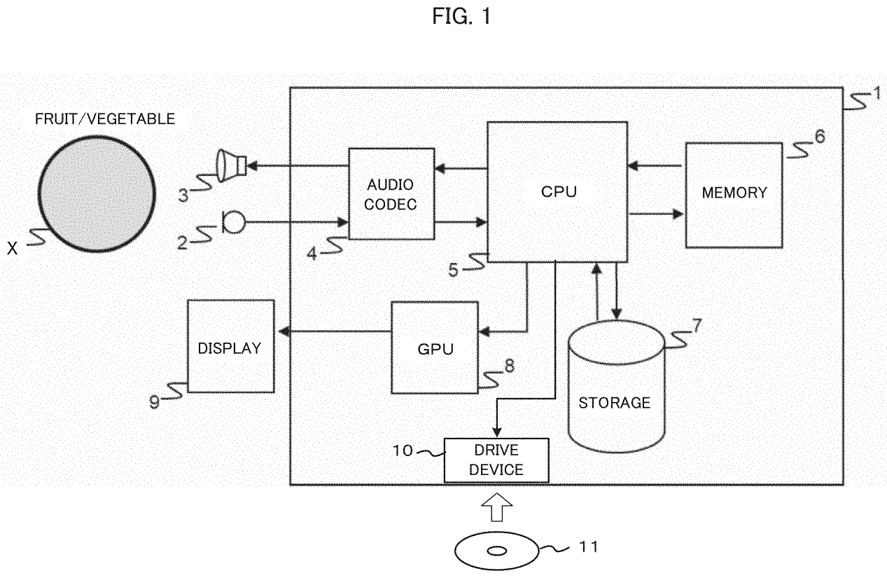

(Configuration of Apparatus) FIG. 1 shows the overall configuration of the inspection apparatus 1 according to the first example embodiment. As an example of a learning type non-destructive inspection apparatus, the inspection apparatus 1 detects the afterripening degree of fruit/vegetable. The fruit/vegetable X is eaten after its afterripening, for example, watermelon, melon, avocado, sweet potato, pumpkin, tomato, western pear, banana, peach, mango, papaya, chelimoya, passion fruit, dolian, and the like.

As shown in FIG. 1, the inspection apparatus 1 includes an audio codec 4, a CPU (Central Processing Unit) 5, a memory 6, a storage 7, a GPU (Graphics Processing Unit) 8, and a drive device 10. The inspection apparatus 1 can be configured by a general computer terminal. The inspection apparatus 1 is connected to a microphone 2, a speaker 3, and a display 9.

The audio codec 4 converts the audio data created by the CPU 5 into an audio signal and supplies the audio signal to the speaker 3, and also converts the audio signal inputted from the microphone 2 into the audio data and supplies the audio data to the CPU 5.

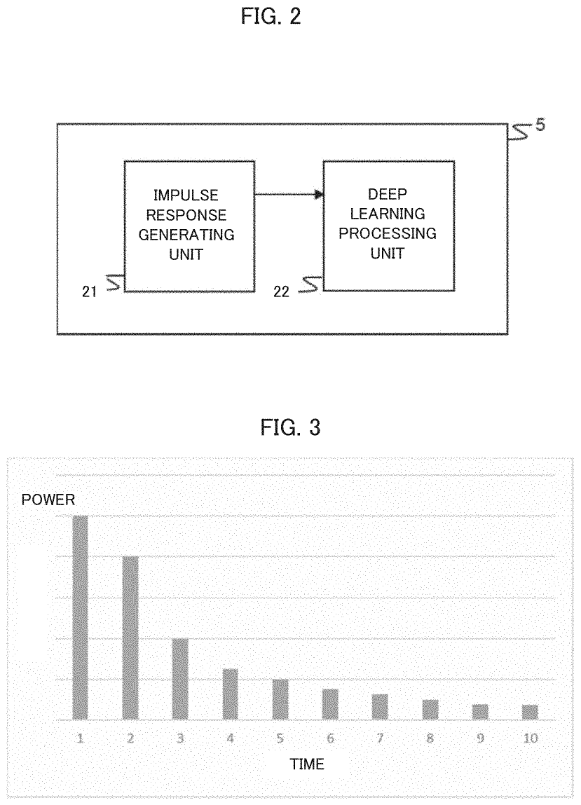

The CPU 5 controls the entire inspection apparatus 1 by executing a program prepared in advance. In particular, the CPU 5 executes the learning process and the determination process described later. FIG. 2 shows the functional configuration of the CPU 5. The CPU 5 functions as an impulse response generating unit 21 and a deep learning processing unit 22 by executing a program prepared in advance.

The memory 6 is composed of a ROM (Read Only Memory), a RAM (Random Access Memory), or the like. The memory 6 temporarily stores various programs to be executed by the CPU 5. The memory 6 is also used as a work memory during the execution of various processes by the CPU 5. The storage 7 stores various types of data necessary for the processes performed by the inspection apparatus 1. Specifically, the storage 7 stores data for generating an acoustic signal to be outputted to the fruit/vegetable, data relating to the neural network for estimating the afterripening degree of the fruit/vegetable by deep learning, teacher data for learning a model composed of the neural network, and the like.

The GPU 8 processes the data outputted from the CPU 5 to generate image data for display and supplies it to the display 9. The display 9 displays information such as the afterripening degree of the fruit/vegetable estimated by the inspection apparatus 1. The drive device 10 reads information from the recording medium 11. The recording medium 11 is a non-volatile, non-transitory recording medium such as a disk-shaped recording medium, a semiconductor memory, or the like, and is configured to be detachable from the inspection apparatus 1. The recording medium 11 records various programs to be executed by the CPU 5. When the inspection apparatus 1 executes the learning process or the determination process, the program recorded on the recording medium 11 is loaded into the memory 6 and is executed by the CPU 5.

(Operation)

Next, the operation of the inspection apparatus 1 will be described. The inspection apparatus 1 applies an acoustic signal to the fruit/vegetable to give an acoustic stimulus, and estimates the afterripening degree of the fruit/vegetable based on the reflected signal of the acoustic signal. Specifically, first, the impulse response generating unit 21 of the CPU 5 generates a swept-sine signal as an acoustic signal, and outputs it to the audio codec 4. The “swept-sine signal”, also called as a swept sinusoidal signal, is a signal whose frequency rises or falls over time and is used to generate an impulse response. The audio codec 4 converts the swept-sine signal into an audio signal, and the speaker 3 outputs the swept-sine signal toward the fruit/vegetable X.

The microphone 2 receives the acoustic signal (also referred to as a “reflected signal”) reflected by the fruit/vegetable X, and supplies it to the audio codec 4. The audio codec 4 converts the reflected signal to a digital signal and supplies it to the CPU 5.

The impulse response generating unit 21 generates an inverse swept-sine signal of the swept-sine signal given to the fruit/vegetable X, and generates an impulse response by performing a convolution operation with the reflected signal received from the audio codec 4. Further, the impulse response generating unit 21 generates a short-time power signal of the generated impulse response. FIG. 3 shows an image of the short-time power signal generated by the impulse response generating unit 21. The short-time power signal is data indicating the power value present at equally spaced time. As for the afterripening degree of the fruit/vegetable, the power of the impulse response obtained from the fruit/vegetable decreases with the passage of time, because the afterripening of the fruit/vegetable advances as time passes. The generated short-time power signal is inputted to the deep learning processing unit 22. Incidentally, the impulse response generating unit 21 is an example of a generation unit, an acquisition unit, and a computation unit.

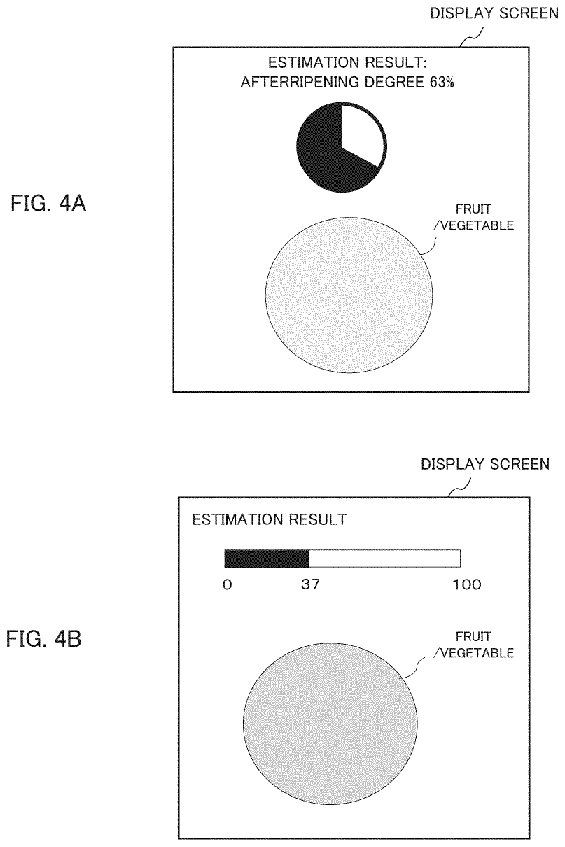

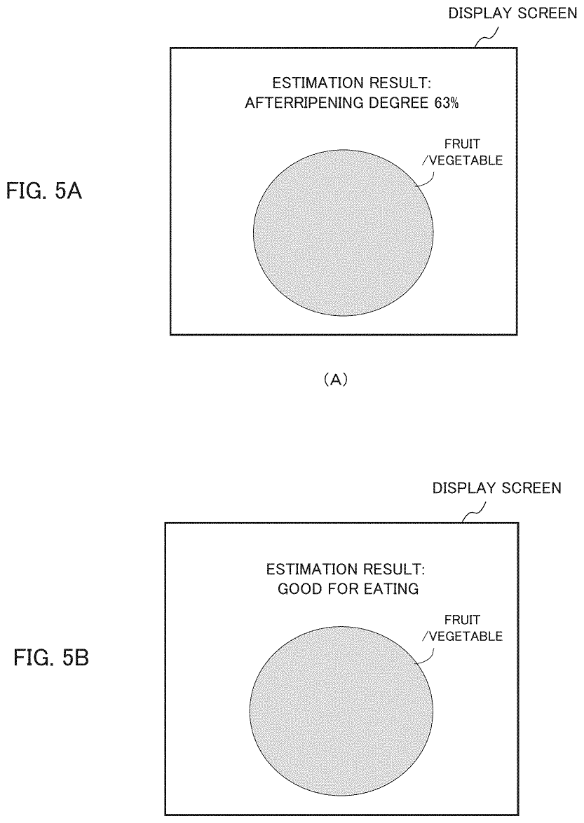

The deep learning processing unit 22 estimates the afterripening degree of the fruit/vegetable X by deep learning. Specifically, the deep learning processing unit 22 performs regression analysis by deep learning using the short-time power signal generated by the impulse response generating unit 21 as an explanatory variable and the degree of ripening of the fruit/vegetable as an objective variable. The deep learning processing unit 22 uses a model composed of a neural network, and this model is learned in advance by a learning process to be described later. Information about the configuration of the model and the parameters obtained by the learning are stored in the storage 7. At the time of estimation, the deep learning processing unit 22 uses the learned model to estimate the afterripening degree. Image data indicating the estimation result by the deep learning processing unit 22 is displayed on the display 9 via the GPU 8. For example, the image data may display the estimation result of the afterripening degree by color of the fruit/vegetable, a pie chart, a bar graph, or the like. FIG. 4A is a display example showing the estimation result in a pie graph, and FIG. 4B is a display example showing the estimation result in a bar graph. Further, the estimation result of the afterripening degree may be displayed by a numerical value (e.g., “afterripening degree 63%), a level (e.g., “6 out of 10 stages”), or the words indicating the afterripening degree (e.g., “good to eat”). FIG. 5A is a display example showing the estimation result in numerical values, and FIG. 5B is a display example showing the estimation result in words. Further, for example, the estimation result of the afterripening degree may be displayed by combining several of the above-described display examples, such as displaying a combination of a numerical value and a color, or displaying a combination of words and a numerical value. Incidentally, the deep learning processing unit 22 is an example of an estimation unit.

Next, the processes executed by the inspection apparatus 1 will be described in detail.

### (Learning Process)

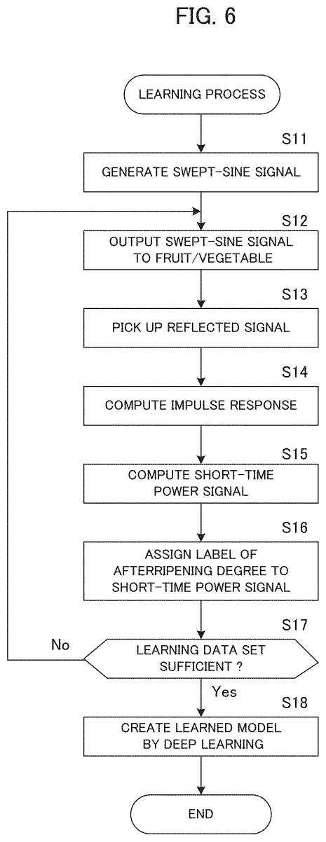

First, a learning process by the inspection apparatus 1 will be described. FIG. 6 is a flowchart of the learning process by the inspection apparatus 1. This process is performed by the CPU 5 executing a program prepared in advance and functioning as the impulse response generating unit 21 and the deep learning processing unit 22.

First, the impulse response generating unit 21 generates a swept-sine signal (step S11). Next, the impulse response generating unit 21 supplies the generated swept-sine signal to the speaker 3 via the audio codec 4, and outputs to the fruit/vegetable X from the speaker 3 (step S12). Next, the microphone 2 picks up the reflected signal from the fruit/vegetable X and supplies it to the impulse response generating unit 21 (step S13).

The impulse response generating unit 21 computes the impulse response by convolving the inverse signal of the swept-sine signal generated in step S1l with the supplied reflected signal (step S14). Next, the impulse response generating unit 21 computes the short-time power signal of the generated impulse response and supplies it to the deep learning processing unit 22 (step S15).

The deep learning processing unit 22 performs regression analysis using the supplied short-time power signal as an explanatory variable, and assigns a label of the afterripening degree to the fruit/vegetable X as an objective variable (step S16). Thus, a set of an impulse response by the fruit/vegetable X and a teacher label of the afterripening degree of the fruit/vegetable corresponding thereto (hereinafter, these are referred to as “learning data sets”) is created.

Next, the deep learning processing unit 22 determines whether or not the learning data set created so far is sufficient (step S17). Specifically, the deep learning processing unit 22 determines whether or not the amount of the learning data set created so far has reached a predetermined amount necessary to sufficiently learn a model for estimating the afterripening degree of the fruit/vegetable X based on the impulse response. When the learning data set is not sufficient (step S17: No), the inspection apparatus 1 repeats steps S12 to S17 to increase the amount of the learning data set. On the other hand, when the learning data set is sufficient (step S17: Yes), the deep learning processing unit 22 performs learning of a model for estimating the afterripening degree of the fruit/vegetable X using the prepared learning data set to create a learned model (step S18). Then, the learning process ends.

(Determination Process)

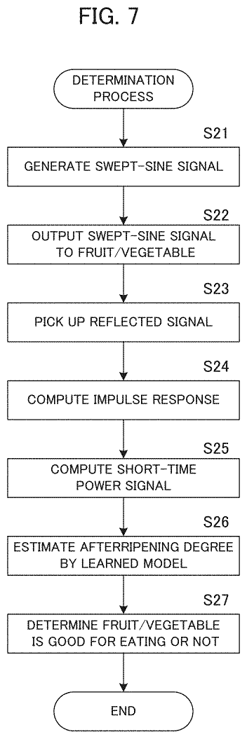

Next, the determination process by the inspection apparatus 1 will be described. FIG. 7 is a flowchart of the determination process by the inspection apparatus 1. This process is also performed by the CPU 5 executing a program prepared in advance and mainly functioning as the impulse response generating unit 21 and the deep learning processing unit 22.

First, the impulse response generating unit 21 performs the same processing as steps S11 to S15 of the learning process. That is, the impulse response generating unit 21 generates a swept-sine signal (step S21), and outputs the generated swept-sine signal from the speaker 3 to the fruit/vegetable X (step S22). Incidentally, the fruit/vegetable X at this time is fruit or a vegetable to be actually determined.

Next, the microphone 2 picks up the reflected signal from the fruit/vegetable X, and supplies it to the impulse response generating unit 21 (step S23). Next, the impulse response generating unit 21 computes the impulse response by convolving the inverse signal of the swept-sine signal generated in step S21 with the supplied reflected signal (step S24), and further computes the short-time power signal of the generated impulse response and supplies it to the deep learning processing unit 22 (step S25).

Thus, when the short-time power signal of the impulse response of the fruit/vegetable X is obtained, the deep learning processing unit 22 estimates the afterripening degree of the fruit/vegetable X. Specifically, the deep learning processing unit 22 performs regression analysis using the learned model created by the learning process, using the short-time power signal obtained in step S25 as the explanatory variable and the afterripening degree of the fruit/vegetable X as the objective variable, and estimates the afterripening degree of the fruit/vegetable X to be judged (step S26). Then, the deep learning processing unit 22 determines whether or not the fruit/vegetable X is good for eating, based on the obtained afterripening degree (step S27). For example, the deep learning processing unit 22 prepares in advance the relationship between the afterripening degree and the good time to eat for each type of fruit/vegetable, and determines the good time to eat the fruit/vegetable X based on the relationship. Then, the process ends.

As described above, in the present example embodiment, the impulse response is computed by applying an acoustic signal to the fruit/vegetable X, and estimates the afterripening degree of the fruit/vegetable X based on the intensity component of the impulse response. Therefore, it is possible to estimate the afterripening degree without causing deterioration or damage to the objective fruit/vegetable. By regularly estimating the afterripening degree of fruit/vegetable, it becomes possible to predict the good time to eat.

(Modification)

While the above example embodiment uses the swept-sine signal to apply the acoustic stimulus, other techniques capable of acquiring impulse responses, such as the M-sequence method, may be used instead. Note that, when using the swept-sine signal, customization to concentrate the power to a particular frequency is required depending on the object.

In the above example embodiment, the afterripening degree is estimated by using the hardness change of the fruit/vegetable. However, the present invention is not limited thereto, and it is applicable to those in which the calibration curve can be created based on the change the impulse response, such as aging of the object.

### Second Example Embodiment

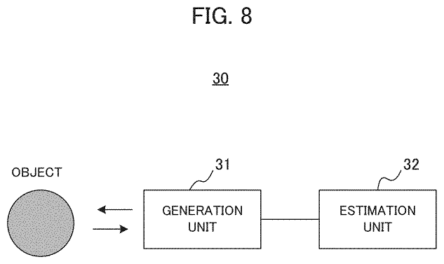

Next, a second example embodiment of the present disclosure will be described. FIG. 8 shows the functional configuration of the state estimation device 30 according to the second example embodiment. As shown, the state estimation device 30 includes a generation unit 31, and an estimation unit 32. The generation unit 31 applies an acoustic stimulus to an object and generates an impulse response of the object. The estimation unit 32 estimates the state of the object by deep learning using the learned model, based on the impulse response. Thus, it is possible to estimate the state of the object without causing deterioration or damage to the object.

While the present invention has been described with reference to the embodiments, the present invention is not limited to the above embodiments. Various changes that can be understood by those skilled in the art within the scope of the present invention can be made in the configuration and details of the present invention. In other words, it is needless to say that the present invention includes various modifications and modifications that could be made by a person skilled in the art according to the entire disclosure, including the scope of the claims, and the technical philosophy. In addition, each disclosure of the above-mentioned patent documents cited shall be incorporated with reference to this document.

This application claims priority based on Japanese Patent Application 2019-173795, filed Sep. 25, 2019, and incorporates all of its disclosure herein by reference.

### DESCRIPTION OF SYMBOLS

1 Inspection apparatus
2 Speaker
3 Microphone
4 Audio codec
5 CPU
6 Memory
7 Storage
21 Impulse response generating unit
22 Deep learning processing unit

What is claimed is:

1. A state estimation device comprising:
a memory configured to store instructions; and
one or more processors configured to execute the instructions to:
apply an acoustic stimulus to an object to generate an impulse response of the object; and
estimate a state of the object based on the impulse response, by deep learning using a learned model.

2. The state estimation device according to claim 1, wherein the one or more processors set an intensity component of the impulse response as an input of a neural network performing the deep learning, and set the state of the object as an output of the neural network.

3. The state estimation device according to claim 1, wherein the one or more processors compute a short-time power signal from the impulse response, use the short-time power signal as an explanatory variable, and estimate the state of the object by performing regression analysis using the state of the object as an objective variable.

4. The state estimation device according to claim 1, wherein the one or more processors are further configured to execute the instructions to:
apply the acoustic signal to the object to acquire a reflected signal of the acoustic signal; and
compute the impulse response of the object to the acoustic signal based on the reflected signal.

5. The state estimation device according to claim 4,
wherein the acoustic signal is a swept-sine signal, and
wherein the one or more processors compute the impulse signal from the reflected signal of the swept-sine signal.

6. The state estimation device according to claim 1,
wherein the object is a vegetable or fruit, and
wherein the state of the object is an afterripening degree of the vegetable or the fruit.

7. A state estimation method comprising:
applying an acoustic stimulus to an object to generate an impulse response of the object; and
estimating a state of the object based on the impulse response, by deep learning using a learned model.

8. A non-transitory computer-readable recording medium recording a program which causes a computer to execute a process of:
applying an acoustic stimulus to an object to generate an impulse response of the object; and
estimating a state of the object based on the impulse response, by deep learning using a learned model.
